(19) United States
(12) Patent Application Publication
(10) Pub. No.: US 2008/0105580 A1
(43) Pub. Date: May 8, 2008

(54) Applicator Tip

(75) Inventors: Brian Nentwick, Derby, CT (US); Roland Ostapoff, Hartford, CT (US)

Correspondence Address: COVIDIEN, 60 MIDDLETOWN AVENUE, NORTH HAVEN, CT, 06473, US

(73) Assignee: TYCO HEALTHCARE GROUP LP, North Haven, CT (US)

(21) Appl. No.: 11/555,878

(22) Filed: Nov. 2, 2006

(51) Int. Cl. A61B 17/06; B65D 35/44; B67D 3/00; G01F 11/00

(52) U.S. Cl. 206/438; 222/1; 222/481; 222/571

(57) ABSTRACT

An applicator tip for selectively dispensing a sterile surgical adhesive from a reservoir and a method for dispensing a sterile surgical adhesive includes a reservoir which contains the adhesive material, an applicator tip with a distal end and an opening offset a distance from the distal end whereby the opening opens to dispense adhesive material when pressure is applied to the applicator tip surface and the applicator tip is in a deformed configuration and whereby adhesive material is not dispensed when pressure is not applied to the applicator tip and the applicator tip is in an un-deformed configuration. A kit includes an applicator tip and a reservoir for dispensing a sterile surgical adhesive within a sealable package.

### BACKGROUND

1. Technical Field

The present disclosure relates generally to devices for the application of sterile compositions, and more particularly to devices and methods for applying surgical sealants or adhesives with an improved applicator tip design.

2. Background of Related Art

Monomer and polymer adhesives are used in both industrial and medical applications. Included among these adhesives are the 1,1-disubstituted ethylene monomers and polymers, such as the α-cyanoacrylates. Medical applications of adhesive compositions include use as an alternate or an adjunct to surgical sutures and staples in wound closure as well as for covering and protecting surface wounds. When such an adhesive is applied, it is usually applied in its monomer form, and the resultant polymerization gives rise to the desired adhesive bond.

However, with the application of adhesive in a monomeric form, due to the rapid polymerization rate of the monomers, it has been challenging to design effective and commercially viable packaging and dispensing systems. Such packaging and dispensing systems must counterbalance the competing requirements that the monomer not prematurely polymerize, that the monomer be easily applied, that the monomer polymerize at a desired rate upon application, and that the sanitary and/or sterile properties of the monomer and applicator be maintained.

Various dispensing and packaging systems for adhesives have been proposed. One of the problems surgeons have faced with such dispensers is the lack of control of the dispensing rate of the adhesive, e.g., large amounts of the adhesive may be inadvertently dispensed and wasted. Furthermore, the adhesive may spill into areas of tissue or onto surfaces where it is not needed and can impede the success of the wound closure.

Further, a problem arises if the dispenser is to be used more than once, as is common. Over multiple uses, contaminants may be transferred from one surface to another surface. As is apparent, this is especially of concern with the application of compositions in the medical field.

Further, there are other problems associated with conventional techniques with the application of adhesive compositions in certain environments, particularly ones in which the surface is difficult to reach or is isolated and where the surgeon will not be able to visualize both the flow of the adhesive as well as the incision.

Another problem arising from known applicator tips is the reactive nature of the material of the tip with the adhesive composition which disrupts the uniform flow of the adhesive and affects the cure rate. Further, a problem arises when the tip clogs due to the non-uniform flow or premature curing of the adhesive.

Accordingly, a need exists for new applicator designs to expel the adhesive without wasting adhesive by filling useless areas of the applicator tips. A need also exists to expel the adhesive in a controlled manner and without expelling through an entire surface area of the tip, and to obtain precise placement of the applicator tip in tissue areas where it is difficult to reach or apply. Further, a need exists for applicator dispensing materials which allow the surgeon to visualize the flow of the adhesive through the applicator tip. Still further, a need exists for dispensing adhesive uniformly without affecting the cure rate and clogging the applicator tip.

Conventional devices fail to provide an applicator and/or an applicator dispensing system that is optimized for convenient dispensing and controlled application of adhesive materials on a variety of surfaces and structures.

### SUMMARY

Accordingly, an applicator device in accordance with the present disclosure is provided to address the need for an easy to use and efficient assembly for dispensing and applying sterile compositions, such as during medical procedures.

In one embodiment, an applicator tip for selectively dispensing a sterile composition contained in a reservoir includes an opening offset from a distal end of the tip which provides a drip free seal in an un-deformed configuration and allows passage of the sterile composition when deformed.

In another embodiment, a method for dispensing a sterile composition includes providing an applicator tip as disclosed in embodiments affixed to a reservoir containing sterile composition and deforming the tip to selectively dispense the sterile composition.

In a still further embodiment, a kit includes a reservoir containing a sterile composition and at least one applicator tip in accordance with the embodiments of this disclosure.

### BRIEF DESCRIPTION OF THE DRAWINGS

Although specific embodiments of the present disclosure will now be described with reference to the drawings, it should be understood that such embodiments are by way of example only and merely illustrative of but a small number of the many possible specific embodiments which can represent applications of the principles of the present disclosure. Various changes and modifications obvious to one skilled in the art to which the present disclosure pertains are deemed to be within the spirit, scope and contemplation of the present disclosure as further defined in the appended claims.

FIG. 1 is a side elevation view of one embodiment of the presently disclosed applicator tip constructed in accordance with illustrative the present disclosure;

FIG. 2 is a bottom plan view of the applicator shown in FIG. 1;

FIG. 3 is a side perspective view of the applicator tip shown in FIG. 1 as positioned on a reservoir;

FIG. 4 is a top plan view of an illustrative kit in accordance with the embodiments of the present disclosure;

FIG. 5 is a side elevation view of the kit embodiment of FIG. 4 in accordance with the embodiments of the present disclosure;

FIG. 6A is a side elevation view of a further illustrative embodiment of the presently disclosed applicator tip constructed in accordance with the present disclosure;

FIG. 6B is a longitudinal cross section taken along section A-A of FIG. 6A;

FIG. 7A is a side elevation view of another illustrative embodiment of the presently disclosed applicator tip constructed in accordance with the present disclosure;

FIG. 7B is a longitudinal cross section taken along section A-A of FIG. 7A;

FIG. 8A is a side elevation view of another illustrative embodiment of the presently disclosed applicator tip constructed in accordance with the present disclosure;

FIG. 8B is a longitudinal cross section taken along section A-A of FIG. 8A;

FIG. 9A is a side elevation view of yet another illustrative embodiment of the presently disclosed applicator tip constructed in accordance with embodiments of the present disclosure;

FIG. 9B is a longitudinal cross section taken along section A-A of FIG. 9A;

FIG. 10A is a side elevation view of a further illustrative embodiment of the presently disclosed applicator tip constructed in accordance with the embodiments of the present disclosure;

FIG. 10B is a longitudinal cross section taken along section A-A of FIG. 10A;

FIG. 11A is a side elevation view of another illustrative embodiment of the presently disclosed applicator tip constructed in accordance with the embodiments of the present disclosure;

FIG. 11B is a longitudinal cross section taken along section A-A of FIG. 11A;

FIG. 12A is a side elevation view of another illustrative embodiment of the presently disclosed applicator tip constructed in accordance with the embodiments of the present disclosure;

FIG. 12B is a longitudinal cross section taken along section A-A of FIG. 12B;

FIG. 13A is a side elevation view of a further illustrative embodiment of the presently disclosed applicator tip constructed in accordance with the embodiments of the present disclosure;

FIG. 13B is a longitudinal cross section taken along section A-A of FIG. 13A;

FIG. 14A is a side elevation view of yet another illustrative embodiment of the presently disclosed applicator tip constructed in accordance with the embodiments of the present disclosure;

FIG. 14B is a longitudinal cross section taken along section A-A of FIG. 14A;

FIG. 15A is a side elevation view of another illustrative embodiment of the presently disclosed applicator tip constructed in accordance with the embodiments of the present disclosure;

FIG. 15B is a longitudinal cross section taken along section A-A of FIG. 15A;

FIG. 16A is a side elevation view of another illustrative embodiment of the presently disclosed applicator tip constructed in accordance with the embodiments of the present disclosure;

FIG. 16B is a longitudinal cross section taken along section A-A of FIG. 16A;

FIG. 17A is a side elevation view of a further illustrative embodiment of the presently disclosed applicator tip constructed in accordance with the embodiments of the present disclosure;

FIG. 17B is a longitudinal cross section taken along section A-A of FIG. 17A;

FIG. 18A is a side elevation view of yet another illustrative embodiment of the presently disclosed applicator tip constructed in accordance with the embodiments of the present disclosure; and

FIG. 18B is a longitudinal cross section taken along section A-A of FIG. 18A.

### DETAILED DESCRIPTION

In general, the present disclosure is directed to an applicator tip for selectively dispensing sterile composition contained in a reservoir, a method for dispensing a sterile composition and a kit comprising the applicator tip for dispensing a sterile composition in a reservoir. While the following description is presented in connection with the delivery of sterile surgical adhesive compositions, it should be understood that the present applicator tip may be employed to dispense any sterile composition, especially those useful in medical procedures. Illustrative examples of sterile compositions that may be dispensed using the present applicator tips include, but are not limited to, adhesives, sealants, antimicrobial compositions, wound healing compositions, diagnostic compositions, anti-adhesion compositions, and the like.

According to the present disclosure, an applicator tip is provided that can be used to more precisely and economically apply adhesive and sealant materials at a desired application site. Applicator tips of the present disclosure can be used on any of a wide variety of applicator devices, such as ampoules, vials, tubes and syringes, are also amenable to use in some embodiments of the present disclosure.

In general, applicator tips for selectively dispensing sterile surgical adhesive contained in a reservoir according to the present disclosure include an opening offset from a distal end of the tip to provide a drip free seal in an un-deformed configuration and allowing passage of the sterile surgical adhesive when deformed. A method for dispensing a sterile surgical adhesive includes providing an applicator tip in accordance the embodiments of the present disclosure affixed to a reservoir containing sterile surgical adhesive and deforming the tip to selectively dispense the sterile surgical adhesive.

In embodiments, the opening is a slit and is positioned no more than 2 mm away from the distal end of the tip, from about 1.3 mm to about 1.8 mm away from the tip and from about 0.8 to about 1.5 away from the tip wherein the distal end of the tip forms an apex. The applicator tip has a width and the opening has a length that is from about 15% to about 100% of the width of the applicator tip.

The applicator tip is generally of a rigid or semi-rigid material, to permit controlled delivery of the adhesive material to the desired application site. Suitable materials for forming the applicator tip include, but are not limited to, natural materials such as cellulose, foams, rubber, cardboard, metal, ceramic, glass, thermoplastics, thermosets, plastics such as butyrate or high density polyethylene, polypropylene, polyester, for example, or any other suitable material. In one embodiment, the applicator tip is made of a non-porous material and has a decreased affinity for the adhesive. The applicator tip may be made of clear silicone which will allow the surgeon to visualize both the flow of the adhesive through the tip as well as the application site. In general, the only limitation on the materials used to fabricate the applicator tips is that the material must be sufficiently compatible with the composition to be dispensed therein, that undesirable effects on the composition do not occur during contact of the composition with the enclosure, and that the applicator tip opening or slit functions according to the present disclosure. Accordingly, the applicator tip will not clog the way current fiber applicators do.

In each of the embodiments, the applicator tip's opening remains closed when the applicator tip is un-deformed or when pressure is not applied to the applicator tip, or alternatively, to the reservoir. This eliminates dripping of the adhesive from the reservoir and allows a surgeon control over the amount of adhesive used in wound closure. In further embodiments, the applicator tip is configured and dimensioned so that the tip may reach hard to reach surfaces and areas.

The applicator tip according to the present disclosure can be used in conjunction with a wide variety of applications of biocompatible solvent composition materials, wherein it is necessary or desirable to efficiently and easily apply an adhesive material. Examples include, but are not limited to, those applicable to medical, industrial, and home use. For example, the applicator tip in accordance with the present disclosure may be used to apply a composition of solvent and moisturizing agent, for wound closure adhesives such as those used for surgically incised or traumatically lacerated tissues; retarding blood flow from wounds; dressing burns; dressing skin or treating stomatitis or other superficial or surface sores or wounds.

The applicator tip may be used to store and apply a wide variety of solvents, including but not limited to: isopropyl alcohol, benzyl alcohol, esters of acetic acid and/or mixtures thereof. The applicator tip may be used with a number of different adhesives including polymerizable liquid adhesives such as 1,1-disubstituted ethylene monomers and polymers, including cyanoacrylate monomers such as the α-cyanoacrylates.

The presently disclosed applicator tip and kit will be further described in conjunction with the accompanying figures showing exemplary embodiments of the present disclosure. In the figures, like numerals have been used to identify like components.

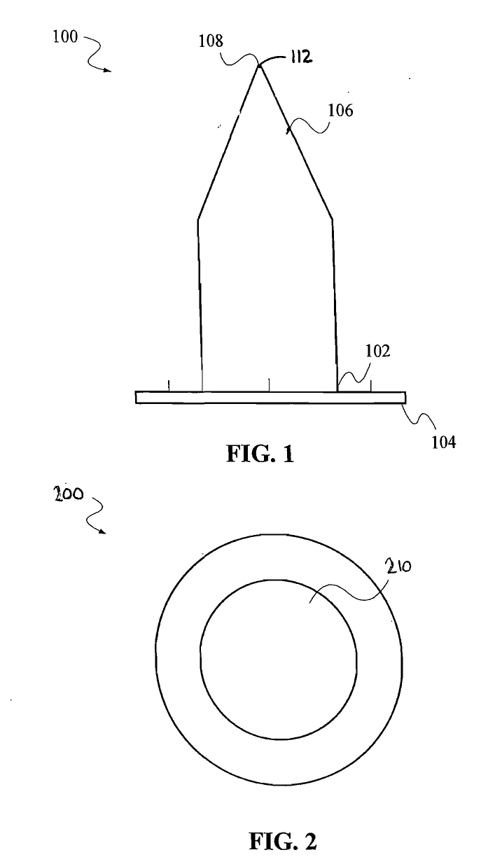

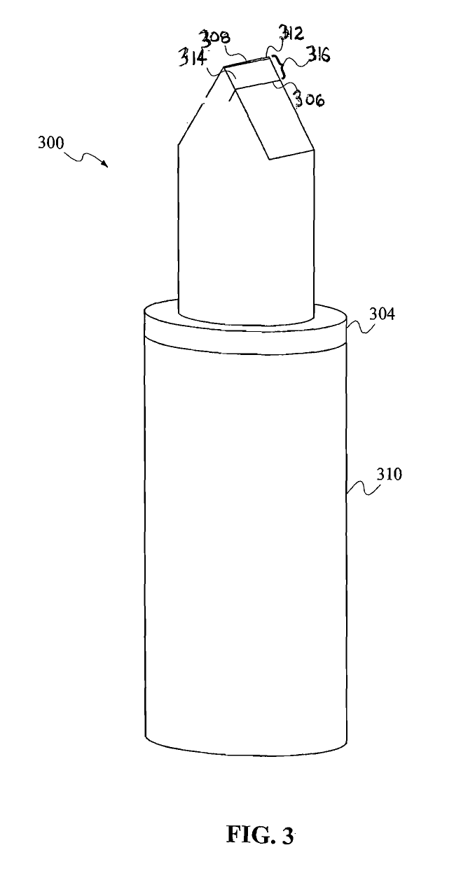

FIG. 1 shows an applicator tip 100 in accordance with an embodiment of the present disclosure for selectively dispensing sterile surgical adhesive contained in a reservoir conveniently, inexpensively and effectively. The applicator tip 100 contains a base portion 104 which includes a proximal end 210 (as shown in FIG. 2), a distal end 108 and opening 114 offset from distal end 108 in an un-deformed configuration. FIG. 3 shows an applicator tip 300 for dispensing adhesive contained in reservoir 310 with an opening 306 offset from distal end 308 which forms an apex 312. Although tip 300 and reservoir 310 are illustrated as separate components, tip 300 may be permanently attached to or formed with body 310. Opening 306 is offset from the distal end 308 of applicator tip 300 a distance 316 on face 314.

The applicator tip 100 may be made of rubber, silicone or any other resilient material. The applicator tip 100 may be any of a variety of shapes and designs dependent on numerous factors including, for example, the specific contents of the container body 310 and the intended use of the adhesive contained within the container body 310. The opening 106 may be configured and dimensioned into a variety of shapes and designs, more specifically into a shape of a slit, rectangle, circle, wedge, star, triangle or trapezoid.

When a user wants to apply the adhesive or sealant, for example, for wound closure, the user applies pressure to the applicator tip 100 along the tissue of a patient, or in the alternative, by applying pressure to reservoir 310 to change the configuration of applicator tip 100 from an un-deformed to a deformed configuration which allows for flow of the adhesive from reservoir 310 via base portion 104 and proximal end 210 and then through opening 106 to the wound. Because of the inherent resilience of applicator tip 100, opening 106 will remain closed and un-deformed when no pressure is applied to applicator tip 100, or in the alternative, when no pressure is applied to reservoir 310. The dimensions of the applicator tip 300 allow it to be used to apply adhesives and sealants in a variety of environments and spatial constraints.

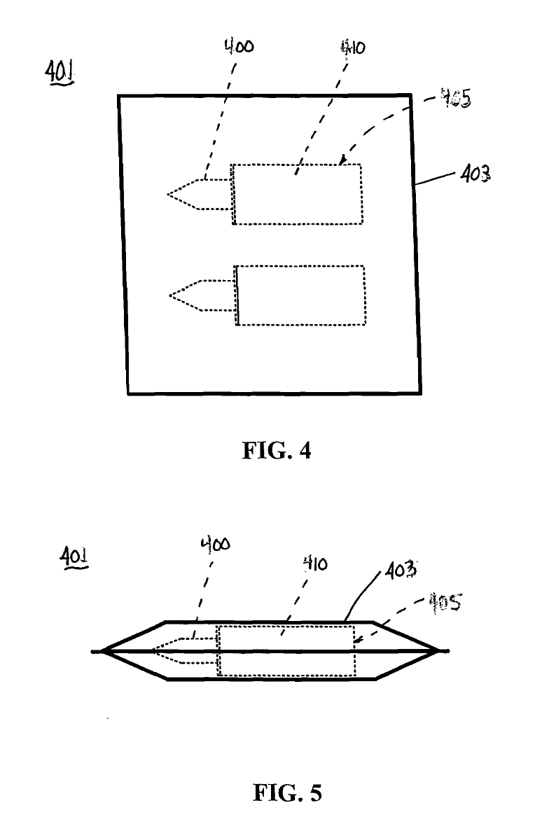

The presently disclosed applicator tip 400 and container body 410 may be included in a kit as shown in FIGS. 4 and 5. The kit may include one or more tips of the same or different sizes, one or more containers for adhesive or the like and an enclosure 403 for housing the various components of the kit. The kit can include any conventional enclosure or sealable pouch used for medical devices manufactured from any suitable material known to those skilled in the art. In one embodiment, an enclosure is formed by heat sealing two panels of aluminum foil coated on the interior surfaces thereof with a heat sealable polymeric composition. Other means for sealing the enclosure may be employed as are well known to those skilled in the art. FIGS. 4 and 5 show kit 401 which includes at least one applicator assembly 405 comprising applicator tip 400 attached to reservoir 410 within an enclosure 403 in accordance with the embodiments of the present disclosure.

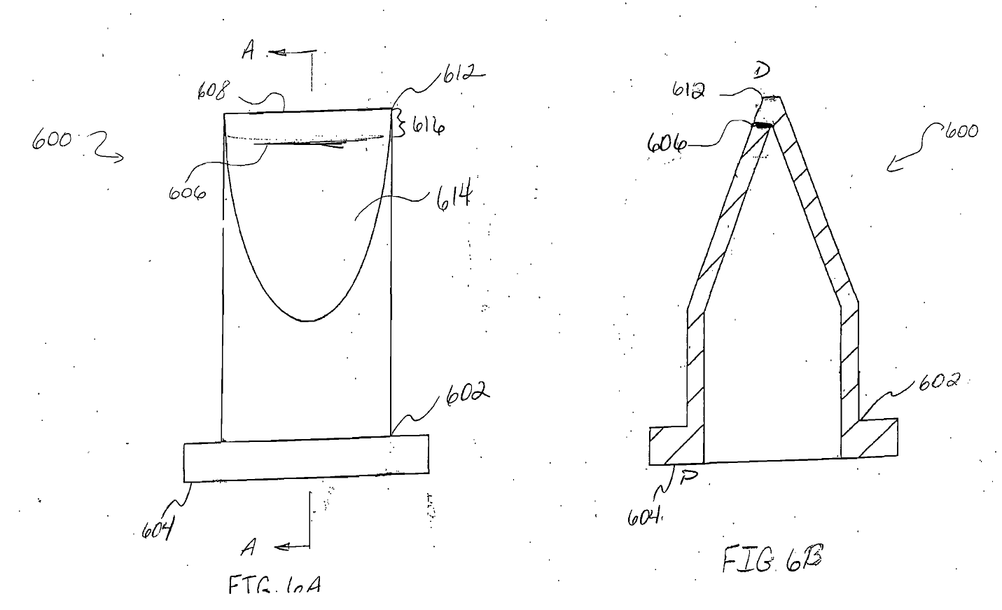

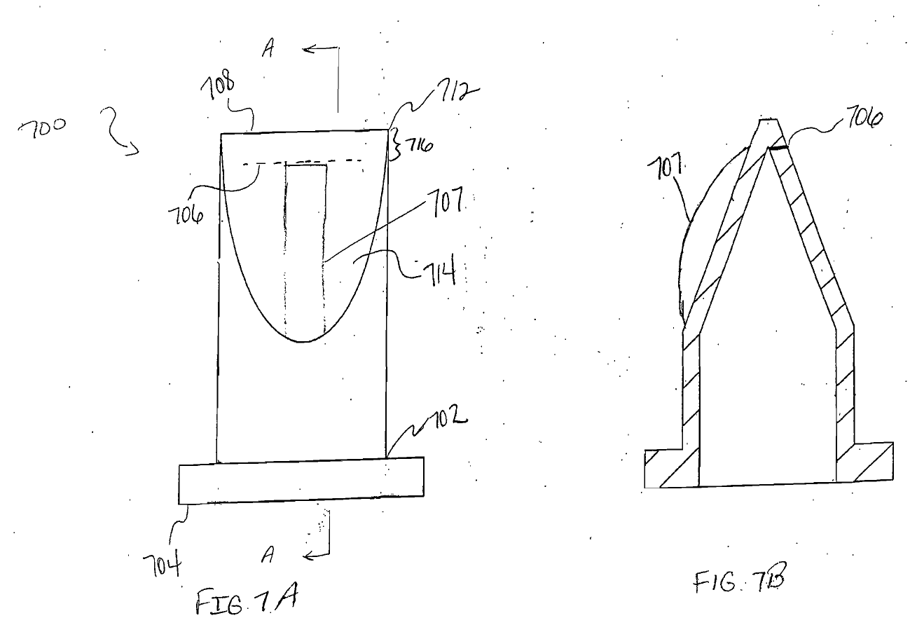

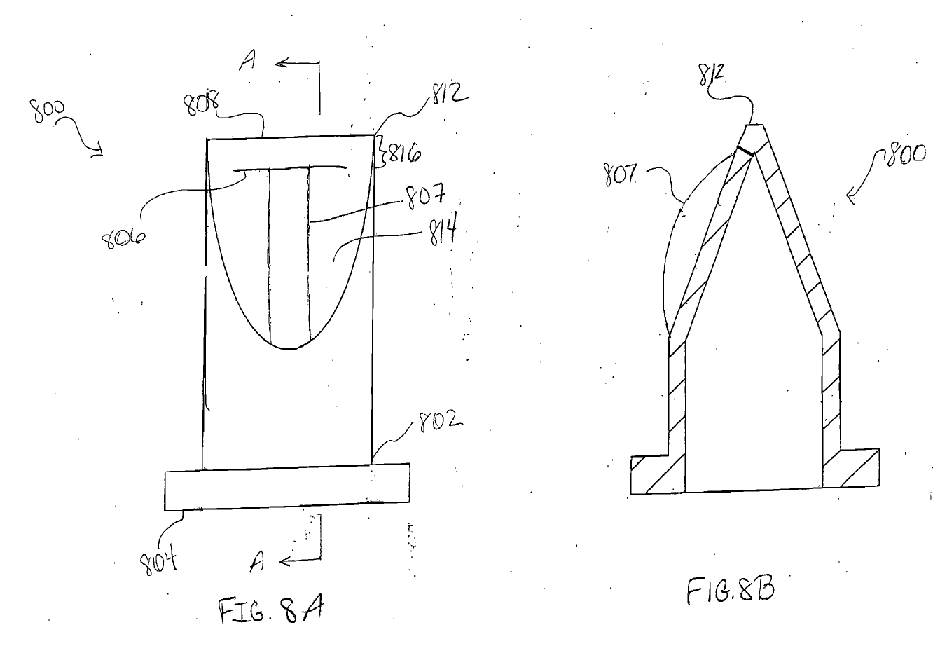

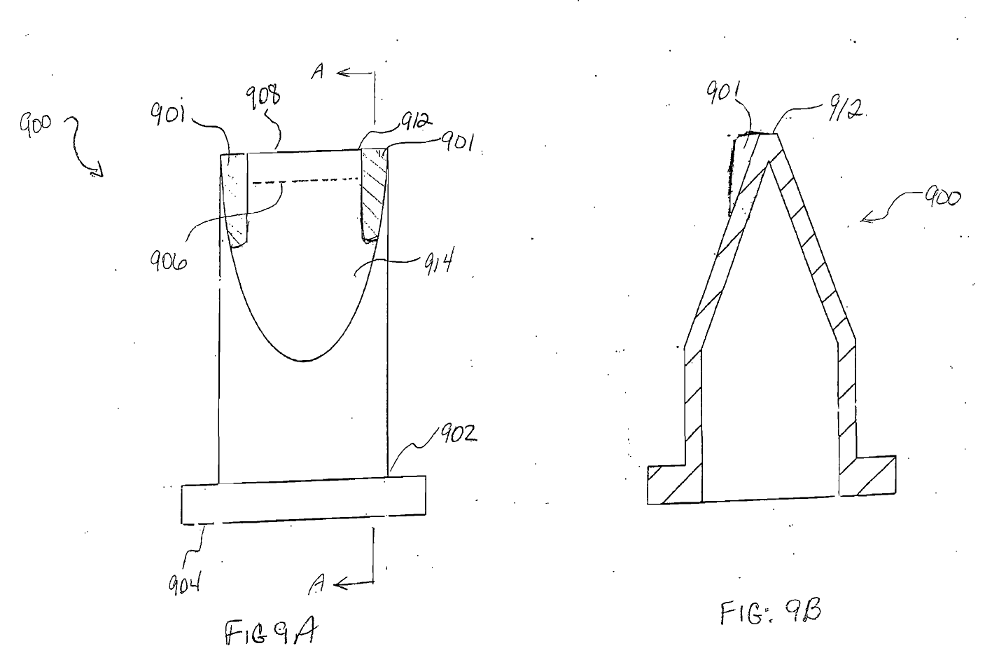

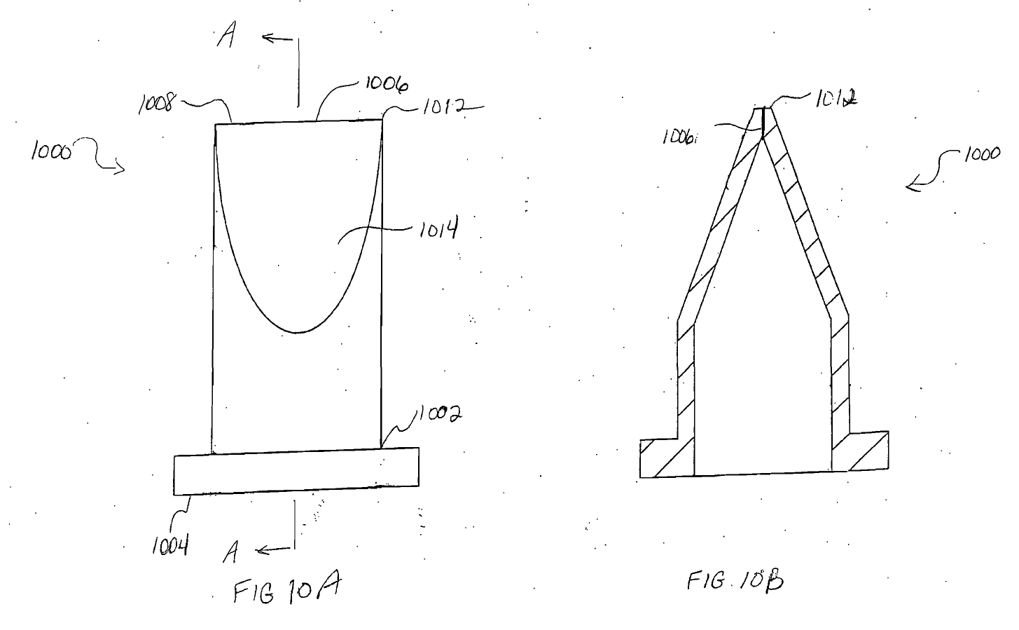

FIGS. 6A-18B are alternative embodiments of the presently disclosed applicator tip in accordance with the embodiments of the present disclosure for selectively dispensing sterile surgical adhesive contained in a reservoir. In FIG. 6A, opening 606 is at distance 616 from distal end 608 of apex 612 on face 614 of applicator tip 600. Opening 606 is positioned no more than 2 mm away from distal end 608 of apex 612 of applicator tip 600 as shown in FIGS. 6A-6B.

In FIGS. 7A-7B, opening 706 is approximately 1 mm from distal end 708 on the opposite face of applicator tip 700 from stripe 707 located on face 714. Stripe 707 extends towards distal end 708 and positioned centrally on applicator tip 700. Opening 706 on the opposite face of face 714 is indicated by a dashed line. Stripe 707 is shown as a center line arc on the face opposite of opening 706 in FIG. 7B. Stripe 707 allows for better dispersion of materials to a wound from applicator tip 700 as it used by the surgeon as a visual aid for pulling across the center of the wound.

In FIGS. 8A-8B, opening 806 is approximately 1 mm from distal end 808 with stripe 807 located centrally on face 814 and extending toward distal end 808. Stripe 807 is shown as a center line arc on the same side or face of opening 806 in FIG. 8B. The smooth geometry of stripe 807 allows for restricted flexing of applicator tip 800 thereby allowing for even distribution of an adhesive via opening 806 of applicator tip 800 to a designated surface.

In FIGS. 9A-9B, opening 906 is approximately 1 mm away from distal end 908 of apex 912 with stripes 901 on an opposite face 914 of opening 906 along each outer edge of applicator tip 900. Stripes 901 are a visual aide to assist the surgeon in more even application of an adhesive material to a wound and provides for more rigid flexing of opening 906 for better distribution of adhesive.

In FIGS. 10A-10B, opening 1006 is located directly on distal end 1008 of apex 1012 of applicator tip 1000. The opening 1006 allows the surgeon to target areas that are not so easily accessible via other means.

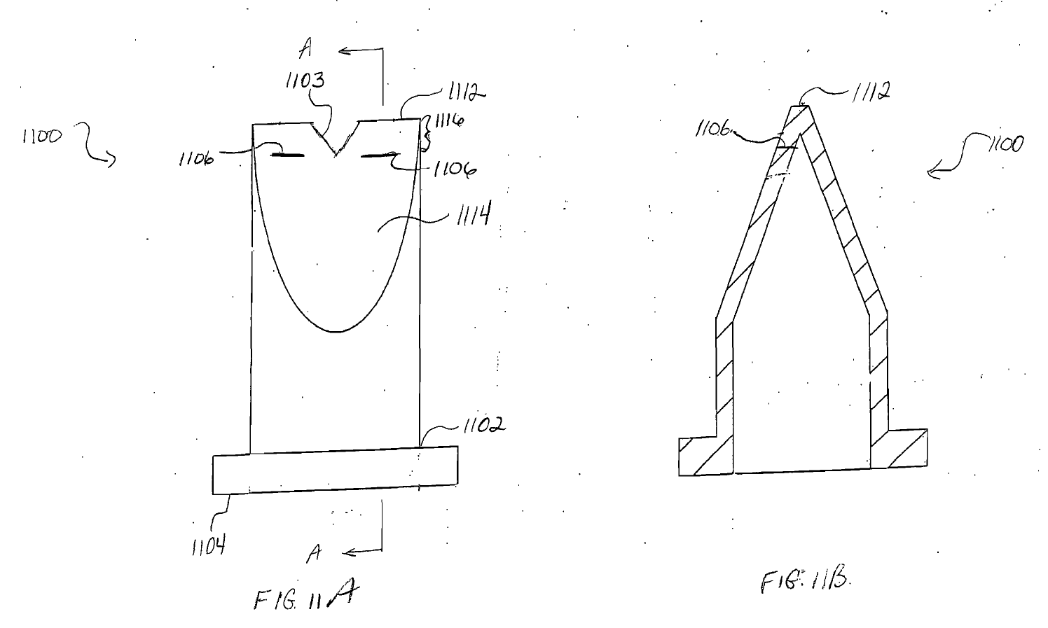

In FIGS. 11A-11B, openings 1106 are at a distance 1116 from distal end 1108 on face 1114 of applicator 1100. Center line v-shaped wedge 1103 extends from distal end 1108 of apex 1112 penetrating both faces of applicator tip 1100. Opening 1106 is not a contiguous opening across face 1114 and v-shaped wedge 1103 but rather comprises two separate openings 1106 which allows for more effective application of adhesive materials for particular wound closures.

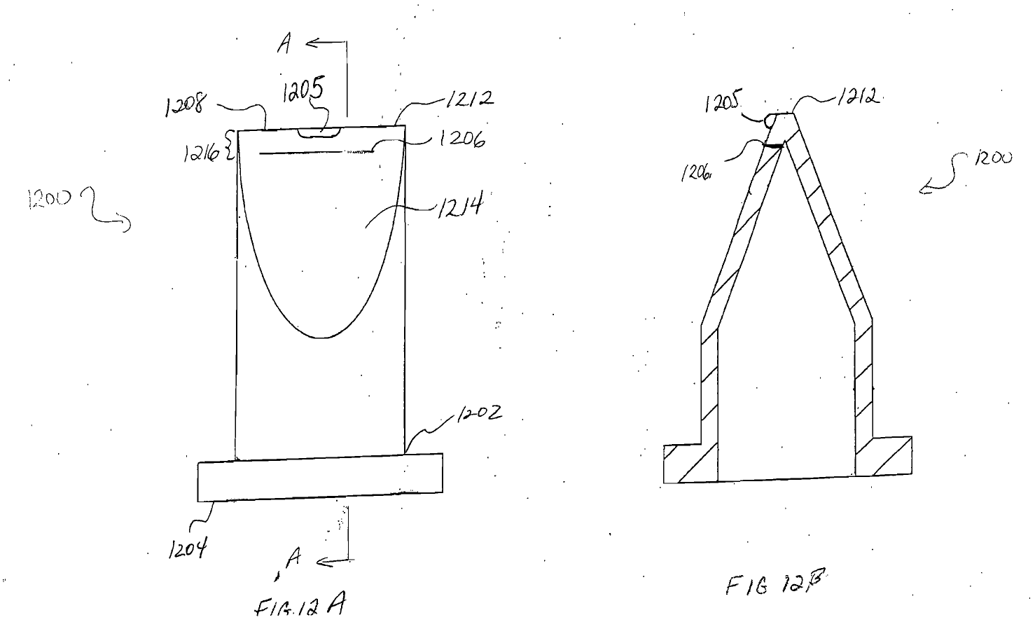

In FIGS. 12A-12B, opening 1206 is at a distance 1216 from distal end 1208 of apex 1212 on face 1214 of applicator 1200. Center line tab 1205 extends from distal end 1208 but is not in contact with opening 1206. Center line tab 1205 is selectively positioned in connection with opening 1206 so as to dispense adhesive from opening 1206 at a desired rate for the specific procedure being performed.

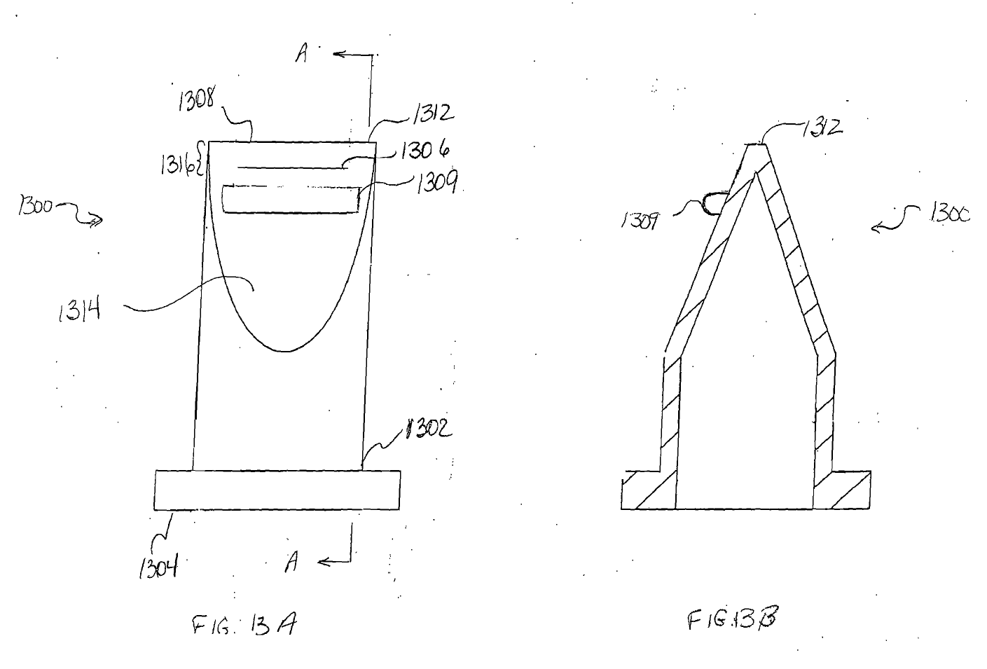

In FIGS. 13A-13B, opening 1306 is at a distance 1316 from distal end 1308 on face 1314 of applicator 1300. Spatula 1309 is located on face 1314 proximal to opening 1306. Spatula 1309 allows for restricted flexing of applicator tip 1300 to control flow of adhesive through opening 1306 thereby allowing for even distribution of the material and for protection of surrounding tissue while the surgeon is applying the material to a wound.

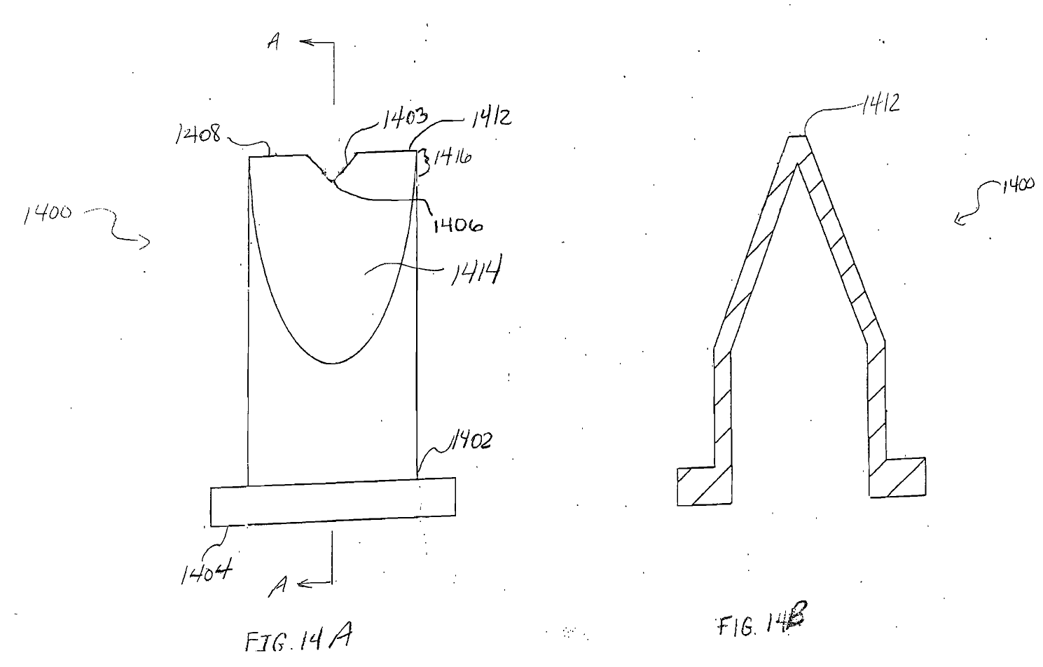

In FIGS. 14A-14B, opening 1406 is at a distance 1416 from distal end 1408 of apex 1412 at the base of v-shaped wedge 1403. V-shaped wedge 1403 extends from distal end 1408 of apex 1412 and penetrates both faces of applicator tip 1400.

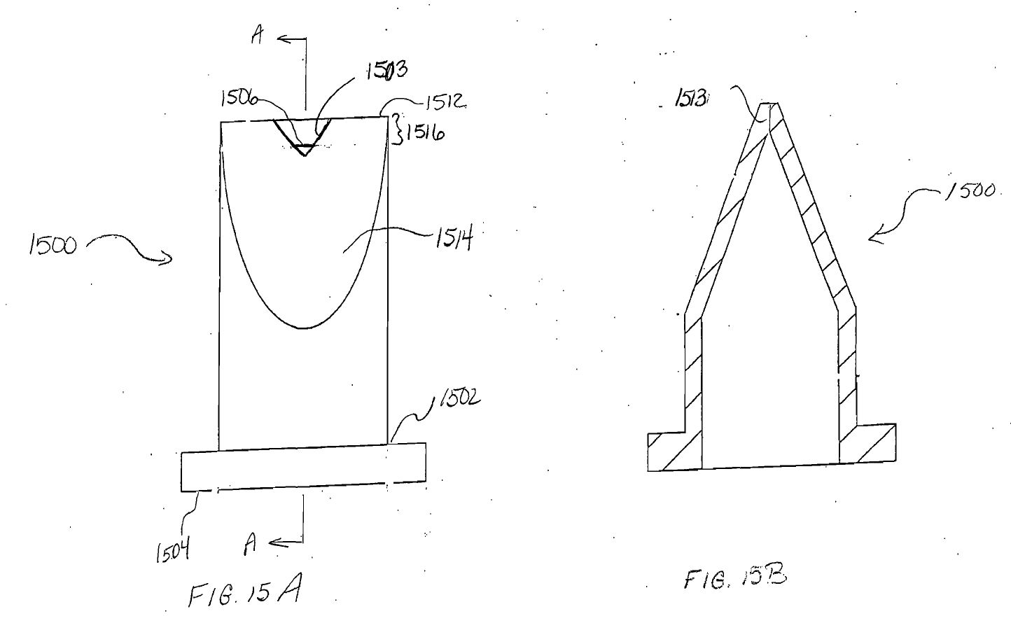

In FIGS. 15A-15B, opening 1506 is at a distance 1516 from distal end 1508 of apex 1512 on face 1514 of applicator 1500. V-shaped wedge 1503 extends from distal end 1508 of apex 1512 penetrating below opening 1506 on face 1514 of applicator tip 1500.

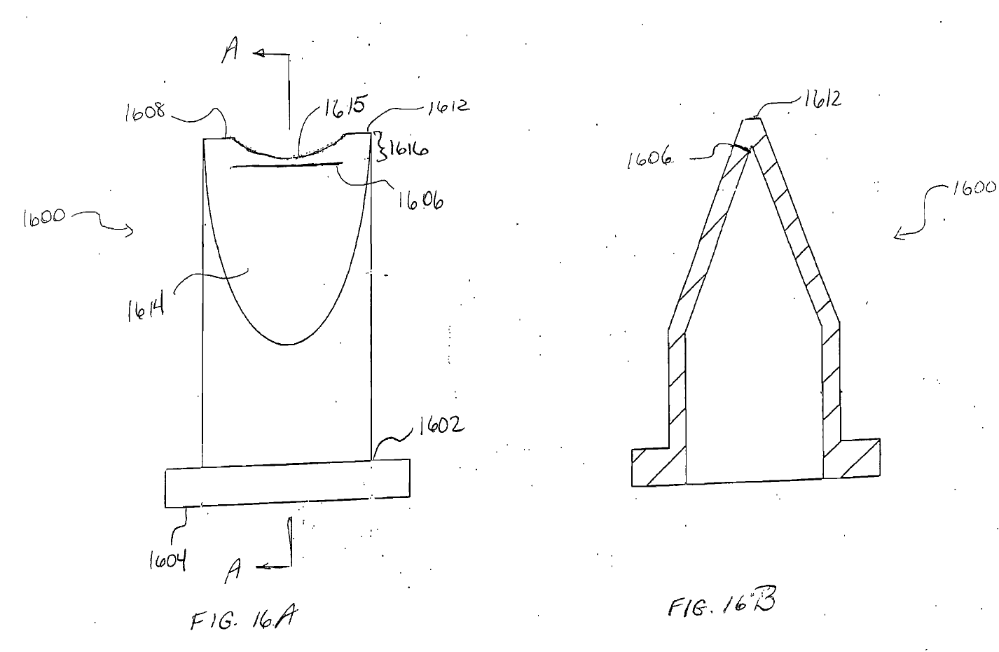

In FIGS. 16A-16B, opening 1606 is at a distance 1616 from distal end 1608 of apex 1612 on face 1614 of applicator 1600. Curved indent 1615 extends from one point of distal end 1608 to another point of distal end 1608 distal to opening 1606.

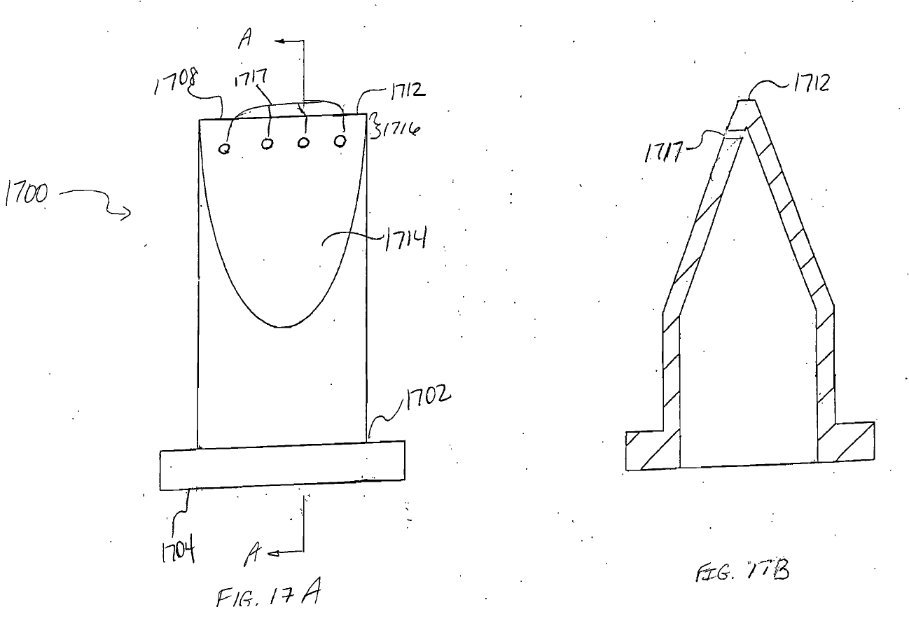

In FIGS. 17A-17B, multiple openings 1717 are at a distance 1716 from distal end 1708 of apex 1712 on face 1714 of applicator tip 1700 from which adhesive will flow through to the intended surface.

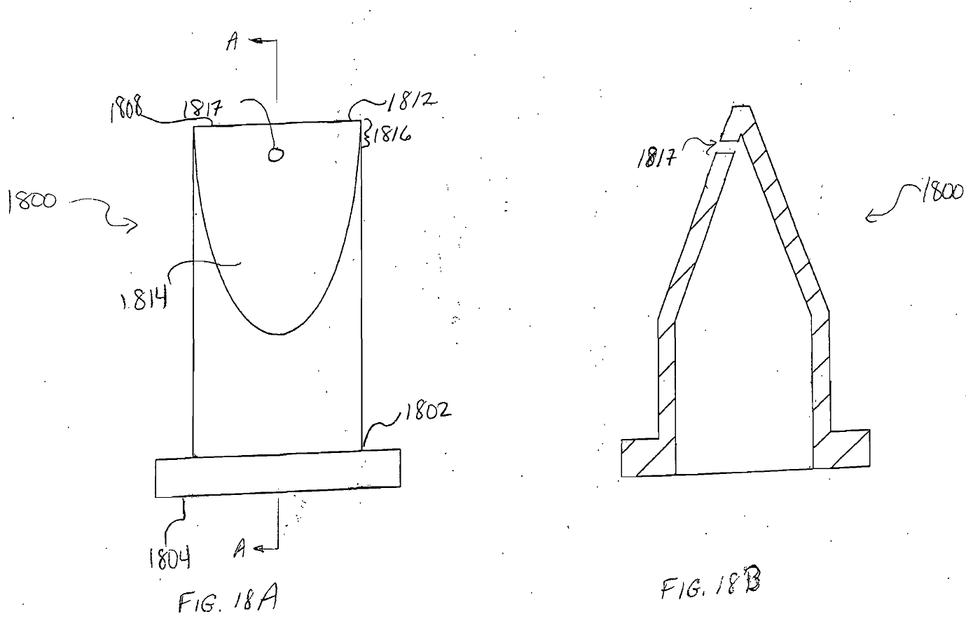

In FIGS. 18A-18B, opening 1817 is at a distance 1816 from distal end 1808 of apex 1812 on face 1814 of applicator tip 1800 from which adhesive will flow through to the intended surface.

It should be recognized that the applicator tip shown in FIGS. 1-18B are only illustrative and not limiting. For example, the applicator may be a wide variety of shapes and sizes such as circular, elliptical, elongated, curved, tapered, chiseled or square depending on the particular area where the adhesive needs to be applied. Also, in alternative illustrative embodiments, the applicator tip could be in the form of a brush, sponge or constructed of foam. Further, the applicator tip may include a cover to protect the sterility of the applicator prior to use.

The adhesive contained in a reservoir may also contain suitable bioactive materials which include, but are not limited to, medicaments such as antibiotics, antimicrobials, antiseptics, bacteriocins, bacteriostats, disinfectants, steroids, anesthetics, fungicides, anti-inflammatory agents, antibacterial agents, antiviral agents, antitumor agents, growth promoting substances, moisturizing components, antioxidants, tackifiers, solubilizers, colorants, perfumes, surfactants, UV absorbers, inorganic fillers and pH adjusting agents or mixtures thereof.

Preferable medicaments are those that are anions or help in radical generation or that are ion pairs or are themselves radicals. In embodiments, the medicament includes, but is not limited to a quaternary ammonium halide such as alkylbenzyldimethylammonium chloride (benzalkonium chloride; BAC) with an alkyl containing 6-18 carbon atoms, its pure components, or mixtures thereof, or benzethonium chloride; or a salt of sulfadiazine, such as a silver, sodium, or zinc salt, water-soluble placenta extract, allantoin, lecithin, amino acids, kojic acid, proteins, saccharides, hormones, placenta extract, components extracted from various types of herbal medicine such as aloe, sponge gourd and liquorice, vitamin A, vitamin C, vitamin D, vitamin E and other vitamins, etc. or mixtures thereof.

With regard to the moisturizing components, an aqueous solution of succinylkefiran, an aqueous solution of acetylkefiran, an aqueous solution of maleylkefiran, malt sprout extract, Rosae fructus extract, orange extract, orange fruit juice, raspberry extract, kiwi extract, cucumber extract, gardenia extract, grapefruit extract, Crataegus cuneata extract, xanthoxylum extract, Crataegus oxycantha extract, Juniperus communis extract, Zizyphi fructus extract, Ziziphus jujuba extract, duke extract, tomato extract, grape extract, sponge gourd extract, lime fruit juice, apple extract, apple fruit juice, lemon extract, lemon fruit juice, etc. can be added singly or in combinations of two or more types.

With regard to the antioxidants, ascorbic acid, propyl gallate, butyl hydroxyanisole, dibutyl hydroxytoluene, nordihydroguaiaretic acid, tocopherol, tocopherol acetate, etc. or mixtures thereof can be added.

With regard to the tackifiers, casein, pullulan, agar, dextran, sodium alginate, soluble starch, carboxy starch, dextrin, carboxymethyl cellulose, sodium carboxymethyl cellulose, methyl cellulose, ethyl cellulose, hydroxyethyl cellulose, polyvinyl alcohol, polyethylene oxide, polyacrylamide, polyacrylic acid, polyvinylpyrrolidone, carboxyvinyl polymer, polyvinyl ether, methyl vinyl ether-maleic anhydride copolymer, isobutylene-maleic anhydride copolymer, polyethyleneimine, etc. can be added.

With regard to the solubilizers, benzyl alcohol, pyrrothiodecane, peppermint oil, isopropyl myristate, crotamiton, etc. or mixtures thereof can be added.

With regard to the colorants, those that can have a large influence on the image of the preparation and contribute to an improvement in the user's feeling during use and a feeling of skin revitalization are preferred, for example, approved colorants such as Red No. 2 (amaranth), Red No. 3 (erythrosine), Red No. 102 (new coccine), Red No. 104 (1) (phloxine B), Red No. 105 (1) (rose bengal), Red No. 106 (acid red), Yellow No. 4 (tartrazine), Yellow No. 5 (sunset yellow FCF), Green No. 3 (fast green FCF), Blue No. 1 (brilliant blue FCF) and Blue No. 2 (indigo carmine) or mixtures thereof can be added, but they are not particularly limited thereby.

With regard to the surfactants, anionic surfactants such as sodium dioctylsulfosuccinate, alkylsulfate salts, 2-ethylhexylalkylsulfate ester sodium salt and sodium n-dodecylbenzenesulfonate, cationic surfactants such as hexadecyltrimethylammonium chloride, octadecyldimethylbenzylammonium chloride and polyoxyethylenedodecylmonomethylammonium chloride, nonionic surfactants such as polyoxyethylene stearyl ether, polyoxyethylene tridecyl ether, polyoxyethylene nonyl phenyl ether, polyoxyethylene octyl phenyl ether, polyoxyethylene monostearate, sorbitan monostearate, sorbitan monopalmitate, sorbitan sesquioleate, polyoxyethylene sorbitan monolaurate, polyoxyethylene sorbitan monooleate, glycerol monostearate, polyglycerol fatty acid esters and polyoxyethylene octadecylamine can be added.

With regard to the UV absorbers, p-aminobenzoic acid, p-aminobenzoate esters, amyl p-dimethylaminobenzoate, salicylate esters, menthyl anthranilate, umbelliferone, esculin, benzyl cinnamate, cinoxate, guaiazulene, urocanic acid, 2-(2-hydroxy-5-methylpheny)benzotriazole, 4-methoxybenzophenone, 2-hydroxy-4-methoxybenzophenone, dioxybenzone, octabenzone, dihydroxydimethoxybenzophenone, sulisobenzone, benzoresorcinol, octyldimethyl p-aminobenzoate, ethylhexyl p-methoxy cinnamate, etc. or mixtures thereof can be added.

With regard to the inorganic fillers, titanium oxide, talc, zinc oxide, hydrated silica, magnesium carbonate, calcium hydrogenphosphate, magnesium silicate, diatomaceous earth, silicic anhydride, bentonite, etc. or mixtures thereof can be added.

With regard to the pH adjusting agents, acetic acid, formic acid, lactic acid, tartaric acid, oxalic acid, benzoic acid, glycolic acid, malic acid, citric acid, hydrochloric acid, nitric acid, sulfuric acid, sodium hydroxide, potassium hydroxide, methylamine, ethylamine, propylamine, dimethylamine, diethylamine, dipropylamine, trimethylamine, triethylamine, tripropylamine, monomethanolamine, monoethanolamine, monopropanolamine, dimethanolamine, diethanolamine, dipropanolamine, trimethanolamine, triethanolamine, tripropanolamine, etc., or mixtures thereof can be added.

Another optional ingredient is selected from essential fatty acids (EFAs), i.e., those fatty acids which are essential for the plasma membrane formation of all cells, in keratinocytes EFA deficiency makes cells hyperproliferative. Supplementation of EFA corrects this. EFAs also enhance lipid biosynthesis of epidermis and provide lipids for the barrier formation of the epidermis. The essential fatty acids are preferably chosen from linoleic acid, Y-linolenic acid, homo-Y-linolenic acid, columbinic acid, eicosa-(n-6,9,13)-trienoic acid, arachidonic acid, Y-linolenic acid, timnodonic acid, hexaenoic acid and mixtures thereof.

Antimicrobial and antifungal actives can be effective to prevent the proliferation and growth of bacteria and fungi. Non-limiting examples of antimicrobial and antifungal actives include antibiotic drugs, quaternary ammonium compounds such as benzalkonium chloride; benzethonium chloride; triclosan; triclocarban; and mixtures thereof and the like. Anti-wrinkle, anti-skin atrophy and skin repair actives can be effective in replenishing or rejuvenating the epidermal layer. These actives generally provide these desirable skin care benefits by promoting or maintaining the natural process of desquamation. Non-limiting examples of anti-wrinkle and anti-skin atrophy actives include retinoic acid and its derivatives, and the like. Skin barrier repair actives are those skin care actives which can help repair and replenish the natural moisture barrier function of the epidermis. Non-steroidal cosmetic soothing actives can be effective in preventing or treating inflammation of the skin. The soothing active enhances the skin appearance benefits of the present disclosure, e.g., such agents contribute to a more uniform and acceptable skin tone or color. Benefit agents in the present disclosure may also include anti-itch ingredients. Suitable examples of anti-itch ingredients which are useful in the compositions of the present disclosure include hydrocortisone, and the like.

Various sterilization processes may be used for the separate components of the package assembly or kit. Examples include, but are not limited to, chemical sterilization (e.g., exposure to ethylene oxide or hydrogen peroxide vapor), physical sterilization (e.g., dry or moist heat) or other techniques such as microwave irradiation, gamma radiation, ionizing radiation, and electron beam irradiation. It will be understood that the same or different sterilization technique may be used to sterilize different components of the package assembly.

According to illustrative embodiments of the present disclosure, sequential sterilization can be performed with substantially no resulting change to the adhesive. Accordingly, the sterilized adhesive compositions can have a satisfactory shelf life.

While the present disclosure presents specific embodiments outlined above, it is evident that many alternatives, modifications and variations may be apparent to those skilled in the art. For example, various different combinations, and shapes, sizes and arrangements, of the described features are contemplated. Accordingly, the embodiments of the present disclosure as set forth herein are intended to be illustrative, not limiting. Various changes may be made without departing from the spirit and scope of the present disclosure.

We claim:

1. An applicator tip for selectively dispensing sterile composition contained in a reservoir, the tip having an opening offset from a distal end thereof, the opening providing a drip free seal in an un-deformed configuration and allowing passage of the sterile composition when deformed.

2. An applicator tip as in claim 1, wherein the opening is a slit.

3. An applicator tip as in claim 1, wherein the distal end of the tip forms an apex.

4. An applicator tip as in claim 1, wherein the opening is positioned no more than 2 mm away from the distal end of the tip.

5. An applicator tip as in claim 1, wherein the tip comprises silicone or rubber.

6. An applicator tip as in claim 1, wherein the tip has a width and the opening has a length that is from about 15% to about 100% of the width of the tip.

7. An applicator tip as in claim 1, wherein the reservoir is selected from the group consisting of ampules, vials, tubes and syringes.

8. An applicator tip as in claim 1, wherein the tip includes a generally v-shaped wedge in the distal end thereof.

9. An applicator tip as in claim 1, wherein the tip includes a plurality of openings offset from a distal end thereof.

10. An applicator tip as in claim 1, wherein the tip further comprises a visual aide on a side of the tip opposite from the opening.

11. An applicator tip as in claim 1, wherein the visual aide comprises a stripe on the tip extending toward the distal end thereof.

12. An applicator tip as in claim 1, wherein the stripe is positioned centrally on the tip.

13. An applicator tip as in claim 1, wherein the visual aide comprises a pair of stripes along each outer edge of the tip.

14. An applicator tip as in claim 1, wherein the tip further comprises a center line tab distal of the opening.

15. An applicator tip as in claim 1, wherein the tip further comprises a spatula proximal of the opening.

16. An applicator tip as in claim 1, wherein the tip further comprises a curved indent distal of the opening.

17. A kit comprising a reservoir containing a sterile composition and an applicator tip as in claim 1.

18. A kit as in claim 17 wherein the sterile composition is selected from the group consisting of adhesives, sealants, antimicrobial compositions, wound healing compositions, diagnostic compositions and anti-adhesion compositions.

19. A method for dispensing a sterile composition comprising; providing an applicator tip in accordance with claim 1 affixed to a reservoir containing sterile composition; and deforming the tip to selectively dispense the sterile surgical adhesive.

20. A method as in claim 19 wherein the sterile composition is selected from the group consisting of adhesives, sealants, antimicrobial compositions, wound healing compositions, diagnostic compositions and anti-adhesion compositions.
